(19) United States
(12) Patent Application Publication
(10) Pub. No.: US 2024/0185960 A1
(43) Pub. Date: Jun. 6, 2024

(54) COMPUTATIONAL FLUID DYNAMICS MODEL AND METHODS OF USE

(71) Applicant: Regeneron Pharmaceuticals, Inc., Tarrytown, NY (US)

(72) Inventors: Aevelina Rahman, Nanuet, NJ (US); Bailey Cooper, White Plains, NY (US); Amardeep Singh Bhupender Bhalla, Montvale, NJ (US)

(21) Appl. No.: 18/517,467

(22) Filed: Nov. 22, 2023

Related U.S. Application Data
(60) Provisional application No. 63/429,778, filed on Dec. 2, 2022.

(51) Int. Cl. G16C 20/30; G16C 20/70

(52) U.S. Cl. CPC G16C 20/30; G16C 20/70

(57) ABSTRACT

The present application provides methods for predicting freeze-thaw profiles across scales and geometries, producing predetermined freeze-thaw profiles in scaled-down experiments using a computational fluid dynamics model, and using scaled-down freeze-thaw profiles to predict at-scale freeze-thaw profiles.

### CROSS-REFERENCE TO RELATED APPLICATIONS

This application claims the benefit of U.S. Provisional Application No. 63/429,778, filed Dec. 2, 2022, which is incorporated by reference herein in its entirety.

### FIELD

The present application relates to methods for optimizing freeze-thaw processes to preserve the quality and stability of compounds. The methods described herein are also useful for optimizing freeze-thaw processes to preserve the quality and stability of pharmaceuticals.

### BACKGROUND

Freeze-thaw processes enable operational flexibility while manufacturing compounds, and specifically, pharmaceuticals to preserve quality attributes. For example, freezing stabilizes bulk pharmaceuticals and reduces the likelihood of contamination by microbes during transportation. Immobilizing protein molecules in a frozen matrix minimizes diffusive collisions that can cause aggregation in pharmaceuticals. Freezing also reduces the rates of degradation reactions, especially reactions involving free water, such as peptide bond hydrolysis and aspartic acid isomerization. Therefore, freezing large volumes of pharmaceuticals in batches enables formulation, fill and finish processes to proceed according to real-time commercial and clinical demands.

Freeze-thaw processes can also adversely affect the quality attributes of compounds, and specifically, pharmaceuticals. Temperatures that are too cold can spontaneously unfold proteins (e.g., cold denaturation), and the freeze-thaw rate can alter the physical and chemical properties of solutions in ways that compromise protein stability. Therefore, optimizing freeze-thaw processes optimizes the quality attributes of pharmaceuticals during the manufacturing process, storage, transportation, and delivery. Experiments can characterize freeze-thaw processes in an array of conditions and determine their effects on the quality attributes of the pharmaceuticals. Experiments should be performed at scale because the characteristics of freeze-thaw processes depend on the scale at which the freeze-thaw processes occur. However, bulk pharmaceuticals may not be available for at-scale studies early in development. Monetary, time and labor costs can also limit the investigation of at-scale freeze-thaw scenarios.

It will be appreciated that a need exists for improved methods of characterizing freeze-thaw processes using an array of conditions across scales and determining their effects on the quality attributes of compounds, and specifically, pharmaceuticals.

### SUMMARY

Optimizing freeze-thaw processes to preserve the quality attributes of compounds, such as pharmaceuticals, and especially biopharmaceuticals, is a crucial issue in the manufacturing process, storage, transportation, and delivery of pharmaceuticals. There exists a need for improved methods of characterizing freeze-thaw processes using a variety of conditions across scales to determine their effects on the quality attributes of the pharmaceuticals. The present application provides methods for predicting the characteristics of freeze-thaw processes using various conditions across scales and replicating the characteristics of freeze-thaw processes in scaled-down freeze-thaw processes. Thus, the effects of freeze-thaw processes on the quality attributes of pharmaceuticals can be determined with minimal monetary, time, and labor costs, even if only a small amount of the pharmaceuticals are available.

The present application provides a method for freezing a solution. In some exemplary embodiments, the method comprises (a) using a computational fluid dynamics model to predict a first freezing profile of an at-scale volume of the solution subjected to first freezing operating conditions, wherein the first freezing profile includes predicted average temperatures of the solution during freezing and total freeze time; (b) using the computational fluid dynamics model to fit a transient temperature boundary equation to the first freezing profile; (c) using the computational fluid dynamics model to predict a set-temperature sequence that produces a predicted second freezing profile of a scaled-down volume of the solution, wherein: (i) the transient temperature boundary equation is a condition for predicting the set-temperature sequence; and (ii) the second freezing profile includes predicted average temperatures of the solution during freezing and total freeze time; and (d) freezing the scaled-down volume of the solution using the set-temperature sequence.

In one aspect, the method further comprises determining at least one quality attribute of the scaled-down volume of the solution after freezing.

In one aspect, the solution comprises a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody.

In one aspect, the method further comprises operating a temperature regulation system using the computational fluid dynamics model to produce the set-temperature sequence for freezing the scaled-down volume.

In one aspect, the method further comprises measuring a temperature of at least one point of interest in the scaled-down volume throughout freezing.

In one aspect, the at-scale volume is between about 0.2 L and about 20 L.

In one aspect, the scaled-down volume is between about 20 mL and about 100 mL.

In one aspect, the at-scale volume is in an at-scale container with a volume of between about 1 L and 20 L.

In one aspect, the scaled-down volume is in a scaled-down container with a volume of between about 30 mL and 100 mL.

In one aspect, the at-scale container is selected from a group comprising a 1 L polycarbonate bottle, a 2 L polycarbonate bottle, a 5 L polycarbonate bottle, a 10 L polycarbonate bottle, a 20 L polycarbonate bottle, a 1 L bag, a 2 L bag, an 8.3 L bag, and a 16.6 L bag.

In one aspect, the scaled-down container is selected from a group comprising a 30 mL bag and a 100 mL bag.

The present application provides a method for thawing a solution. In some exemplary embodiments, the method comprises (a) using a computational fluid dynamics model to predict a first thawing profile of an at-scale volume of the solution subjected to first thawing operating conditions, wherein the first thawing profile includes predicted average temperatures of the solution during thawing and total thaw time; (b) using the computational fluid dynamics model to fit a transient temperature boundary equation to the first thawing profile; (c) using the computational fluid dynamics model to predict a set-temperature sequence that produces a predicted second thawing profile of a scaled-down volume of the solution, wherein: (i) the transient temperature boundary equation is a condition for predicting the set-temperature sequence; and (ii) the second thawing profile includes predicted average temperatures of the solution during thawing and total thaw time; and (d) thawing the scaled-down volume of the solution using the set-temperature sequence.

In one aspect, the method further comprises determining at least one quality attribute of the scaled-down volume of the solution after thawing.

In one aspect, the solution comprises a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody.

In one aspect, the method further comprises operating a temperature regulation system using the computational fluid dynamics model to produce the set-temperature sequence for thawing the scaled-down volume.

In one aspect, the method further comprises measuring a temperature of at least one point of interest in the scaled-down volume throughout thawing.

In one aspect, the at-scale volume is between about 0.2 L and about 20 L.

In one aspect, the scaled-down volume is between about 20 mL and about 100 mL.

In one aspect, the at-scale volume is in an at-scale container with a volume of between about 1 L and 20 L.

In one aspect, the scaled-down volume is in a scaled-down container with a volume of between about 30 mL and 100 mL.

In one aspect, the at-scale container is selected from a group comprising a 1 L polycarbonate bottle, a 2 L polycarbonate bottle, a 5 L polycarbonate bottle, a 10 L polycarbonate bottle, a 20 L polycarbonate bottle, a 1 L bag, a 2 L bag, an 8.3 L bag, and a 16.6 L bag.

In one aspect, the scaled-down container is selected from a group comprising a 30 mL bag and a 100 mL bag. The present application provides a method for freezing a solution. In some exemplary embodiments, the method comprises (a) using a computational fluid dynamics model to predict a freezing profile of the at-scale solution subjected to a set of freezing operating conditions; (b) determining if freezing will occur within a necessary period of time; and (c) freezing the at-scale volume of the solution using the set of freezing operating conditions.

The present application provides a method for freeze-thawing a solution. In some exemplary embodiments, the method comprises (a) using a computational fluid dynamics model to predict a first freezing and thawing profile of an at-scale volume of the solution subjected to a first freezing and thawing operating conditions, wherein the first freezing and thawing profile includes predicted average temperatures of the solution during freezing and thawing and total freeze and thaw times; (b) using the computational fluid dynamics model to fit a transient temperature boundary equation to the first freezing and thawing profile; (c) using the computational fluid dynamics model to predict a set-temperature sequence that produces a predicted second freezing and thawing profile of a scaled-down volume of the solution, wherein: (i) the transient temperature boundary equation is a condition for predicting the set-temperature sequence; and (ii) the second freezing and thawing profile includes predicted average temperatures of the solution during freezing and thawing and total freeze and thaw times; and (d) freezing and thawing the scaled-down volume of the solution using the set-temperature sequence.

In one aspect, the method further comprises determining at least one quality attribute of the scaled-down volume of the solution after freezing and thawing.

In one aspect, the solution comprises a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody.

In one aspect, the method further comprises operating a temperature regulation system using the computational fluid dynamics model to produce the set-temperature sequence for freezing and thawing the scaled-down volume.

In one aspect, the method further comprises measuring a temperature of at least one point of interest in the scaled-down volume throughout freezing and thawing.

In one aspect, the at-scale volume is between about 0.2 L and about 20 L.

In one aspect, the scaled-down volume is between about 20 mL and about 100 mL.

In one aspect, the at-scale volume is in an at-scale container with a volume of between about 1 L and 20 L.

In one aspect, the scaled-down volume is in a scaled-down container with a volume of between about 30 mL and 100 mL.

In one aspect, the at-scale container is selected from a group comprising a 1 L polycarbonate bottle, a 2 L polycarbonate bottle, a 5 L polycarbonate bottle, a 10 L polycarbonate bottle, a 20 L polycarbonate bottle, a 1 L bag, a 2 L bag, an 8.3 L bag, and a 16.6 L bag.

In one aspect, the scaled-down container is selected from a group comprising a 30 mL bag and a 100 mL bag.

The present application provides a method for freezing and thawing a solution. In some exemplary embodiments, the method comprises (a) using a computational fluid dynamics model to predict a freezing and thawing profile of the at-scale solution subjected to first freezing and thawing operating conditions; (b) determining if freezing and thawing will occur within a necessary period of time; and (c) freezing and thawing the at-scale volume of the solution using first freezing and thawing operating conditions.

The present application provides a method for thawing a solution. In some exemplary embodiments, the method comprises (a) using a computational fluid dynamics model to predict a thawing profile of the at-scale solution subjected to first thawing operating conditions; (b) determining if thawing will occur within a necessary period of time; and (c) thawing the at-scale volume of the solution using first thawing operating conditions.

The present application provides a method for freezing a solution. In some exemplary embodiments, the method comprises (a) using a computational fluid dynamics model to predict a freezing profile of the at-scale solution subjected to first freezing operating conditions; (b) determining if freezing will occur within a necessary period of time; and (c) freezing the at-scale volume of the solution using first freezing operating conditions.

In one embodiment, the computational fluid dynamics model can account for environmental factors. Environmental factors can include, for example, airflow, proximity to other surfaces of varying temperatures, relative humidity, pressure, and any combinations thereof.

### BRIEF DESCRIPTION OF THE DRAWINGS

FIG. 1 illustrates the outline of the computational fluid dynamics model framework according to exemplary embodiments.

FIG. 2 shows models of an at-scale solution within an at-scale container and a scaled-down solution within a scaled-down container that were produced by a computational fluid dynamics model of the present disclosure according to an exemplary embodiment.

FIG. 3 shows the spatial domain within an at-scale container partitioned into discrete volumes after spatial discretization according to an exemplary embodiment.

FIG. 4 shows predicted thermodynamic temperatures within a scaled-down solution at a point during a freezing process according to an exemplary embodiment.

FIG. 5 shows predicted velocities within an at-scale container partially filled with a solution at a point during a freeze-thaw process according to an exemplary embodiment.

FIG. 6 shows predicted thermodynamic temperatures within an at-scale container partially filled with an at-scale solution at two points during a freezing process according to an exemplary embodiment.

FIG. 7 shows an exemplary embodiment in which a computational fluid dynamics model of the present disclosure can determine whether 3.5 L of a solution in a 5 L Nalgene™ Polycarbonate Biotainer™ Bottle containing a high or low concentration of a drug substance or a free drug substance can freeze in freezing operating conditions within 48 hours.

FIG. 8 shows predicted thermodynamic temperatures and last point to freeze (e.g. the red sphere) a scaled-down solution in a scaled-down container at a point during a freezing process according to an exemplary embodiment.

FIG. 9 shows that weighted spatial averaging of at-scale freeze-thaw processes, generation of a set-temperature sequence for a temperature regulation system, and predictions of the expected temperature profiles of at-scale and scaled-down freeze-thaw processes performed by a computational fluid dynamics model of the present disclosure enable scaled-down freeze-thaw processes to represent the spatially averaged volume of at-scale freeze-thaw processes across scales and geometries according to an exemplary embodiment.

FIG. 10 shows an example of a transient equation fit by a computational fluid dynamics model of the present disclosure to the predicted temperatures of an at-scale solution during a freezing process, set as the transient temperature boundary condition while predicting a temperature progression scheme for a temperature regulation system in a scaled-down experiment intended to reproduce the freeze-thaw rates of at-scale processes, and that regulates a temperature regulation system to produce the predicted temperature progression scheme during a scaled-down experiment according to an exemplary embodiment.

FIG. 11 shows an exemplary embodiment in which the computational fluid dynamics model can approximate the physical quantities of freeze-thaw processes for an at-scale and scaled-down solution in a 1 L-5 L polycarbonate bottle and a 30 mL bag, respectively, and reproduce the freeze-thaw profile of the at-scale solution in the scaled-down solution using a Benchtop Freezing Platform.

FIG. 12 shows the total freeze times of a solution in an at-scale and scaled-down container to the total freeze times predicted by the computational fluid dynamics model according to an exemplary embodiment.

### DETAILED DESCRIPTION

It can be difficult but essential to maintain the quality and stability of compounds, and specifically, biopharmaceuticals during the manufacturing process, storage, transportation, and patient administration. Bulk drug substances go through a series of processing steps to turn a purified drug substance into the final dosage form in an appropriate container closure system or delivery device (e.g., formulation, fill and finish processes). Formulation, fill and finish processes include freezing and thawing of a bulk pharmaceutical substance (e.g., bulk freeze-thaw), formulation of the desired concentration of a purified pharmaceutical substance with excipients, filtration, filling into a container closure system, freeze-drying (if necessary), inspection, labeling & packaging, storage, transport, and delivery (e.g., patient administration). Consequently, biopharmaceuticals are vulnerable to numerous chemical and physical instability sources during the formulation, fill and finish processes that could compromise their efficacy.

Cold temperatures can cause proteins to spontaneously unfold (e.g., cold denaturation) by weakening the hydrophobic effect. A Gibbs free energy function can explain the cold denaturation temperature with an inverted parabolic shape. The negative free energy of unfolding favors thermal denaturation at temperatures greater and less than high- and low-temperature thresholds, respectively. For example, the hydrophobic effect stabilizes a spherical globule protein with an inner hydrophobic core, despite the preferences of polar and nonpolar residues for alternative geometric locations. Thus, lowering the temperature decreases the stabilization provided by the hydrophobic effect and causes cold denaturation below a low-temperature threshold. In addition to absolute temperature, the rate at which freezing occurs can adversely affect protein stability by altering the physical and chemical properties of a solution.

Slow freezing rates may exclude proteins and excipients from the ice-liquid interface, leading to an increase in the concentration of excipients and proteins in the liquid close to the ice crystals (e.g., cryoconcentration). Such an increase in the concentration of excipients can alter the structure of proteins. Increasing the concentration of excipients close to the ice crystals may alter the pH and destabilize proteins because the less soluble buffer components may precipitate. Furthermore, the increased protein concentration increases the likelihood of aggregation and precipitation. Like slow freezing rates, slow thawing rates can stress and damage proteins. Tiny ice crystals may recrystallize during a slow thawing process, and proteins may denature at the ice-liquid interface.

A cryoconcentration created during freezing can again destabilize proteins during thawing. Thus, faster thawing processes that minimize recrystallization and cryoconcentration are typically preferred. For example, mixing processes sufficient to homogenize the solution without shearing and denaturing proteins at the air-liquid interface can speed up the thawing process without negatively affecting product quality. Similarly, faster freezing rates can reduce cryoconcentration; however, fast freezing processes can compromise protein stability.

Fast freezing rates expose proteins to a large ice-liquid interface by forming tiny ice crystals that adsorb proteins on their surface. Proteins concentrating on the surface of crystals at the ice-liquid interfaces can partially unfold and aggregate. Additionally, fast freezing rates can entrap air in the ice, and subsequent thawing can denature proteins at the air-liquid interfaces. Therefore, during biopharmaceutical manufacturing, carefully evaluating and optimizing freeze-thaw process parameters prevents bulk freeze-thaw processes from compromising the quality of drug substances.

Measuring the temperature of a solution at one or more points of interest during freeze-thaw processes can determine the freeze-thaw profiles. These data can determine the average temperature and rates of freezing or thawing of the solution for the duration of the freezing or thawing process. Large scales magnify the formation of cryoconcentrations. Therefore, experiments examine cryoconcentration formation during at-scale freeze-thaw processes. Thus, examining the effect of various freeze-thaw operating conditions on the quality attributes of a pharmaceutical protein product in at-scale freeze-thaw processes can require an extensive amount of physical materials and labor. However, a large amount of a pharmaceutical protein product may not be available during the early development stages, precluding the ability to optimize freeze-thaw operating conditions for at-scale manufacturing processes, storage, and transportation.

Disclosed herein are methods for predicting the freeze-thaw rates of an at-scale solution subjected to a set of freeze-thaw operating conditions, reproducing the at-scale volume of the solution freeze-thaw rates using the scaled-down solution, and determining the effects of the freeze-thaw rates on the quality attributes of the scaled-down volume of the solution. An example described below demonstrates that the total freeze time of an at-scale freezing process differed from the prediction of a computational fluid dynamics model of the present disclosure by about 3.57%. Additionally, the computational fluid dynamics model of the present disclosure reproduced the freezing rates of an at-scale freezing process in a scaled-down freezing experiment. The actual and predicted total freeze times of the scaled-down freezing process differed from the total freeze time of the at-scale freezing process by about 2.86% and about 2.38%, respectively. Consequently, the methods of the present disclosure can be used to determine the effects of at-scale freeze-thaw process on the quality attributes of a pharmaceutical without the costs of performing at-scale investigations. The present disclosure also provides a method for determining if a set of freezing operating conditions is suitable for freezing or thawing a volume of a solution within a predetermined amount of time.

Unless described otherwise, all technical and scientific terms used herein have the same meaning as commonly understood by one of ordinary skill in the art to which this invention belongs. Although practice or testing can use methods and materials similar or equivalent to those described herein, particular methods and materials are now described.

The terms “a” and “an” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

In exemplary embodiments, the disclosure provides a method for freezing or thawing a solution. A solution may comprise, for example, a component with at least one quality attribute that freezing or thawing processes can preserve, replicate or predict, a pharmaceutical or pharmaceutical product, an active ingredient, a pharmaceutical drug, biotherapeutics, small-molecule drugs, a drug substance, an excipient or any combination thereof. A pharmaceutical drug may be a pharmaceutical formulation comprising an excipient.

As used herein, the term “composition” refers to a pharmaceutical product formulated together with one or more pharmaceutically acceptable vehicles.

As used herein, the terms “pharmaceutical” and “pharmaceutical product” can include a biologically active component of a drug product. A pharmaceutical and a pharmaceutical product can refer to any substance or combination of substances used in a drug product, intended to furnish pharmacological activity or to otherwise have a direct or indirect effect on the diagnosis, cure, mitigation, treatment, or prevention of disease, or to have a direct or indirect effect in restoring, correcting or modifying physiological functions in animals. Non-limiting methods to prepare a pharmaceutical and a pharmaceutical product can include using a fermentation process, recombinant DNA, isolation and recovery from natural resources, chemical synthesis, biosynthesis, polymerase chain reaction, or combinations thereof. In some exemplary embodiments, a pharmaceutical and a pharmaceutical product is a drug, a chemical compound, a nucleic acid, a nucleotide, a nucleoside, an oligonucleotide, a toxin, a peptide, a protein, a fusion protein, an antibody, an antibody fragment, a Fab region of an antibody, an antibody-drug conjugate, or a pharmaceutical protein product, or combinations thereof.

As used herein, the terms “protein” and “pharmaceutical protein product” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid-phase peptide synthesis methods are known to those of skill in the art. A protein may comprise one or multiple polypeptides to form a single functioning biomolecule. A protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect baculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 Biotechnology and Genetic Engineering Reviews 147-176 (2012), the entire teachings of which are herein incorporated). Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, a protein and a pharmaceutical protein product can be a recombinant protein, an antibody, a bispecific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG (e.g., IgG1, IgG2, IgG3, IgG4), IgM, IgA1, IgA2, IgD, or IgE. In certain exemplary embodiments, the antibody molecule is a full-length antibody (e.g., an IgG1 or IgG4 immunoglobulin), or the antibody can be a fragment (e.g., an Fc fragment or a Fab fragment).

The term “antibody,” as used herein, includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3.

Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, an F(ab′)2 fragment, an scFv fragment, an Fv fragment, a dsFv diabody, a dAb fragment, an Fd′ fragment, an Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single-chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains, each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated).

As used herein, “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific can also be addressed by the system and method disclosed herein.

The term “monoclonal antibody” as used herein, is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art, including the use of hybridoma, recombinant, and phage display technologies, or a combination thereof.

In some exemplary embodiments, a protein and a pharmaceutical protein product can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some exemplary embodiments, a composition can be used for the treatment, prevention, and/or amelioration of a disease or disorder. Exemplary, non-limiting diseases and disorders that can be treated and/or prevented by the administration of the pharmaceutical formulations of the present invention include, infections; respiratory diseases; pain resulting from any condition associated with neurogenic, neuropathic or nociceptic pain; genetic disorder; congenital disorder; cancer; herpetiformis; chronic idiopathic urticarial; scleroderma, hypertrophic scarring; Whipple's Disease; benign prostate hyperplasia; lung disorders, such as mild, moderate or severe asthma, allergic reactions; Kawasaki disease, sickle cell disease; Churg-Strauss syndrome; Grave's disease; pre-eclampsia; Sjogren's syndrome; autoimmune lymphoproliferative syndrome; autoimmune hemolytic anemia; Barrett's esophagus; autoimmune uveitis; tuberculosis; nephrosis; arthritis, including chronic rheumatoid arthritis; inflammatory bowel diseases, including Crohn's disease and ulcerative colitis; systemic lupus erythematosus; inflammatory diseases; HIV infection; AIDS; LDL apheresis; disorders due to PCSK9-activating mutations (gain of function mutations, “GOF”), disorders due to heterozygous Familial Hypercholesterolemia (heFH); primary hypercholesterolemia; dyslipidemia; cholestatic liver diseases; nephrotic syndrome; hypothyroidism; obesity; atherosclerosis; cardiovascular diseases; neurodegenerative diseases; neonatal Onset Multisystem Inflammatory Disorder (NOM ID/CINCA); Muckle-Wells Syndrome (MWS); Familial Cold Autoinflammatory Syndrome (FCAS); familial Mediterranean fever (FMF); tumor necrosis factor receptor-associated periodic fever syndrome (TRAPS); systemic onset juvenile idiopathic arthritis (Still's Disease); diabetes mellitus type 1 and type 2; auto-immune diseases; motor neuron disease; eye diseases; sexually transmitted diseases; tuberculosis; disease or condition which is ameliorated, inhibited, or reduced by a VEGF antagonist; disease or condition which is ameliorated, inhibited, or reduced by a PD-1 inhibitor; disease or condition which is ameliorated, inhibited, or reduced by a Interleukin antibody; disease or condition which is ameliorated, inhibited, or reduced by a NGF antibody; disease or condition which is ameliorated, inhibited, or reduced by a PCSK9 antibody; disease or condition which is ameliorated, inhibited, or reduced by a ANGPTL antibody; disease or condition which is ameliorated, inhibited, or reduced by an activin antibody; disease or condition which is ameliorated, inhibited, or reduced by a GDF antibody; disease or condition which is ameliorated, inhibited, or reduced by a Fel d 1 antibody; disease or condition which is ameliorated, inhibited, or reduced by a CD antibody; disease or condition which is ameliorated, inhibited, or reduced by a C5 antibody or combinations thereof.

In some exemplary embodiments, a composition can be administered to a patient. Administration may be via any route acceptable to those skilled in the art. Non-limiting routes of administration include oral, topical, or parenteral. Administration via certain parenteral routes may involve introducing the formulations of the present invention into the body of a patient through a needle or a catheter, propelled by a sterile syringe or some other mechanical device such as a continuous infusion system. A composition may be administered using a syringe, injector, pump, or any other device recognized in the art for parenteral administration. A composition may also be administered as an aerosol for absorption in the lung or nasal cavity. The solutions may also be administered for absorption through the mucus membranes, such as in buccal administration.

In some exemplary embodiments, a formulation can further comprise excipients including, but not limited to, buffering agents, bulking agents, tonicity modifiers, solubilizing agents, and preservatives. Other additional excipients can also be selected based on function and compatibility with the formulations may be found, for example, in Remington: the science and practice of pharmacy, (2005); U. S. Pharmacopeia: National formulary; Louis Sanford Goodman et al., Goodman & Gilmans, The Pharmacological Basis of Therapeutics (2001); Kenneth E. Avis, Herbert A. Lieberman & Leon Lachman, Pharmaceutical dosage forms: parenteral medications (1992); Praful Agrawala, Pharmaceutical Dosage Forms: Tablets. Volume 1, 79 Journal of Pharmaceutical Sciences 188 (1990); Herbert A. Lieberman, Martin M. Rieger & Gilbert S. Banker, Pharmaceutical dosage forms: disperse systems (1996); Myra L. Weiner & Lois A. Kotkoskie, Excipient toxicity and safety (2000), herein incorporated by reference in their entirety.

As used herein, the volume of an “at-scale solution” can have a volume of between about 0.2 L and about 100 L, between about 1 L and about 75 L, between about 2 L and about 50 L, between about 5 L and about 25 L, about 0.2 L, about 0.5 L, about 1 L, about 2 L, about 3 L, about 4 L, about 5 L, about 7.5 L, about 10 L, about 15 L, about 16.6 L, about 20 L, about 25 L, about 30 L, about 40 L, about 50 L, about 60 L, about 70 L, about 75 L, about 80 L, about 90 L, or about 100 L.

An at-scale solution can be in a container with a volume of between about 0.2 L and about 100 L, between about 1 L and about 75 L, between about 2 L and about 50 L, between about 5 L and about 25 L, about 0.2 L, about 0.5 L, about 1 L, about 2 L, about 3 L, about 4 L, about 5 L, about 7.5 L, about 8.3 L about 10 L, about 15 L, about 16.6 L, about 20 L, about 25 L, about 30 L, about 40 L, about 50 L, about 60 L, about 70 L, about 75 L, about 80 L, about 80 L, or about 100 L. (e.g., an at-scale container).

As used herein, the volume of a “scaled-down solution” can be between about 1 mL and about 200 mL, between about 5 mL and about 100 mL, between about 10 mL and 75 mL, between about 25 mL and about 50 mL, about 1 mL, about 2 mL, about 3 mL, about 4 mL, about 5 mL, about 7.5 mL, about 10 mL, about 15 mL, about 20 mL, about 25 mL, about 30 mL, about 40 mL, about 50 mL, about 60 mL, about 70 mL, about 75 mL, about 80 mL, about 90 mL, about 100 mL, about 125 mL, about 150 mL, about 175 mL, or about 200 mL.

A scaled-down solution can be in a container with a volume of between about 30 mL and about 250 mL, between about 50 mL and about 200 mL, between about 75 mL and about 175 mL, between about 100 mL and about 150 mL, about 30 mL, about 40 mL, about 50 mL, about 75 mL, about 100 mL, about 125 mL, about 150 mL, about 200 mL, or about 250 mL. (e.g., a scaled-down container).

As used herein, a “freezing profile” refers to any quantitative measure or measures of a freezing process that can be used to quantitatively determine at least one freezing rate. Exemplary embodiments quantitatively determine freezing rates throughout a freezing process until a solution is frozen. In some embodiments, a freezing rate can be directly measured. In some embodiments, a freezing rate can be indirectly determined by measuring at least one physical quantity of a freezing process. Exemplary, non-limiting examples of a quantitative measure that can be used to determine a thawing rate include thermodynamic temperature, electrical conductivity, osmolarity, osmolality, evaporation, condensation, direct or indirect observation of crystalline formation, density, flow, viscosity, and any combination thereof.

As used herein, a “freezing rate” refers to the rate at which a solution decreases in thermodynamic temperature. For example, a freezing rate may be the instantaneous rate at which the thermodynamic temperature of a point of a solution decreases during a freezing process, the instantaneous rate at which the averaged thermodynamic temperature of a volume of a solution decreases during a freezing process, the average rate at which the thermodynamic temperature of a point of a solution decreases throughout a freezing process (e.g., from the point in time a solution is initially subjected to freezing conditions until the point in time the entirety of a solution is initially frozen), or the average rate at which the averaged thermodynamic temperature of a volume of a solution decreases throughout a freezing process (e.g., from the point in time a solution is initially subjected to freezing conditions until the point in time the entirety of a solution is initially frozen).

As used herein, a “thawing profile” refers to any quantitative measures of a thawing process that can be used to quantitatively determine at least one thawing rate. Exemplary embodiments quantitatively determine thawing rates throughout a thawing process until a solution is thawed. In some embodiments, a thawing rate can be directly measured. In some embodiments, a thawing rate can be indirectly determined by measuring at least one physical quantity of a thawing process. Exemplary, non-limiting examples of a quantitative measure that can be used to determine a thawing rate include thermodynamic temperature, electrical conductivity, osmolarity, osmolality, evaporation, condensation, direct or indirect observation of crystalline melting, density, flow, viscosity, and any combination thereof.

As used herein, a “thawing rate” refers to the rate at which a solution increases in thermodynamic temperature. For example, a thawing rate may be the instantaneous rate at which the thermodynamic temperature of a point of a solution increases during a thawing process, the instantaneous rate at which the averaged thermodynamic temperature of a volume of a solution increases during a thawing process, the average rate at which the thermodynamic temperature of a point of a solution increases throughout a thawing process (e.g., from the point in time a solution is initially subjected to thawing conditions until the point in time the entirety of a solution is initially thawed), or the average rate at which the averaged thermodynamic temperature of a volume of a solution increases throughout a thawing process (e.g., from the point in time a solution is initially subjected to thawing conditions until the point in time the entirety of a solution is initially thawed).

As used herein, “freezing operating conditions” refers to the thermal and mechanical properties of material inputs within freezing systems and the operating conditions (e.g., freezing operating conditions) of freezing systems that can affect freezing processes. Exemplary, non-limiting freezing operating conditions that may be included in a freezing operating conditions include temperature, convection mode, volume of a solution, mechanical properties of a solution, thermal properties of a solution, geometry of a solution, volume of a container, mechanical properties of a container, thermal properties of a container, geometry of a container, air between a fill level and the upper boundary of the container, adjacent airflow mechanism, proximity of a container to other containers in a freezing environment, and any combination thereof.

As used herein, “thawing operating conditions” refer to the thermal and mechanical properties of material inputs within thawing systems and the operating conditions (e.g., thawing operating conditions) of thawing systems that can affect thawing processes. Exemplary, non-limiting thawing operating conditions that may be included in thawing operating conditions include temperature, convection mode, volume of a solution, mechanical properties of a solution, thermal properties of a solution, geometry of a solution, volume of a container, mechanical properties of a container, thermal properties of a container, geometry of a container, air between a fill level and the upper boundary of the container, adjacent airflow mechanism, proximity of a container to other containers in a thawing environment, and any combination thereof.

As used herein, a “transient temperature boundary equation” is an equation fit to the predicted averaged thermodynamic temperature of an at-scale solution during a freeze-thaw process. Predicting the freeze-thaw profile of an at-scale solution can provide the predicted averaged thermodynamic temperature of an at-scale solution during a freeze-thaw process throughout the freeze-thaw process (e.g., the transient thermodynamic temperature of a solution during a freeze-thaw process). A computational fluid dynamics model of the present disclosure can replicate the predicted averaged thermodynamic temperature of an at-scale solution during a freeze-thaw process in a scaled-down solution by predicting and subjecting a scaled-down solution to a set-temperature sequence. A computational fluid dynamics model of the present disclosure can set a transient temperature boundary equation as a boundary condition while predicting a set-temperature progression that reproduces the freeze-thaw profile of an at-scale solution in a scaled-down solution. Thus, a transient temperature boundary equation informs a computational fluid dynamics model of the present disclosure about the averaged thermodynamic temperature a set-temperature progression must produce in a scaled-down solution at each point during a freeze-thaw process to reproduce an at-scale freeze-thaw profile.

It is understood that the present invention is not limited to any of the aforesaid solution(s), composition(s), pharmaceutical(s), pharmaceutical product(s), protein(s), pharmaceutical protein product(s), protein(s), polypeptide(s), synthetic polypeptide(s), recombinant protein(s), antibody(ies), antigen-binding portion(s), antigen-binding fragment(s), antibody fragment(s), bispecific antibody(ies), multispecific antibody(ies), formulation(s), excipient(s), cell(s), at-scale solution(s), scaled-down solution(s), freezing profile(s), freezing rate(s), thawing profile(s), thawing rate(s), freezing operating conditions, thawing operating conditions, or transient temperature boundary equation(s) and solution(s), composition(s), pharmaceutical(s), pharmaceutical product(s), protein(s), pharmaceutical protein product(s), protein(s), polypeptide(s), synthetic polypeptide(s), recombinant protein(s), antibody(ies), antigen-binding portion(s), antigen-binding fragment(s), antibody fragment(s), bispecific antibody(ies), multispecific antibody(ies), formulation(s), excipient(s), cell(s), at-scale solution(s), scaled-down solution(s), freezing profile(s), freezing rate(s), thawing profile(s), thawing rate(s), freezing operating conditions, thawing operating conditions, or transient temperature boundary equation(s) can be selected by any suitable means.

The existing method for determining the freeze-thaw rates of at-scale freeze-thaw processes and the effects thereof on the quality attributes of a pharmaceutical requires a large amount of pharmaceutical material. Problematically, producing the necessary amounts of pharmaceutical material for at-scale testing requires significant monetary, labor, material resources, time, and other resources. Accordingly, resource limitations may prohibit the optimization of at-scale freeze-thaw operating conditions. In contrast, exemplary embodiments of the present disclosure can provide location-specific modeling of pharmaceutical freeze-thaw processes in containers across scales and geometries within an array of freeze-thaw operating conditions. Furthermore, exemplary embodiments of the present disclosure can reproduce the freeze-thaw rates of at-scale pharmaceutical freeze-thaw processes in scaled-down experiments. Therefore, the effects of at-scale freeze-thaw rates on the quality attributes of pharmaceuticals can be determined with a minimal amount of pharmaceutical material. Consequently, exemplary embodiments of the present disclosure enable a more rigorous and cost-effective optimization of at-scale pharmaceutical freeze-thaw operating conditions than existing methods.

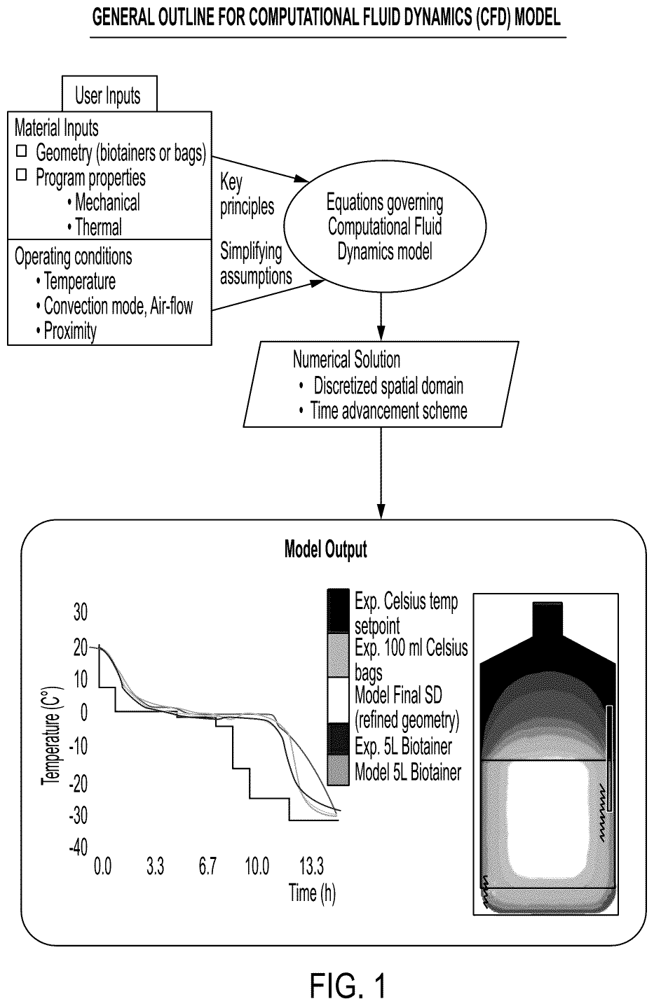

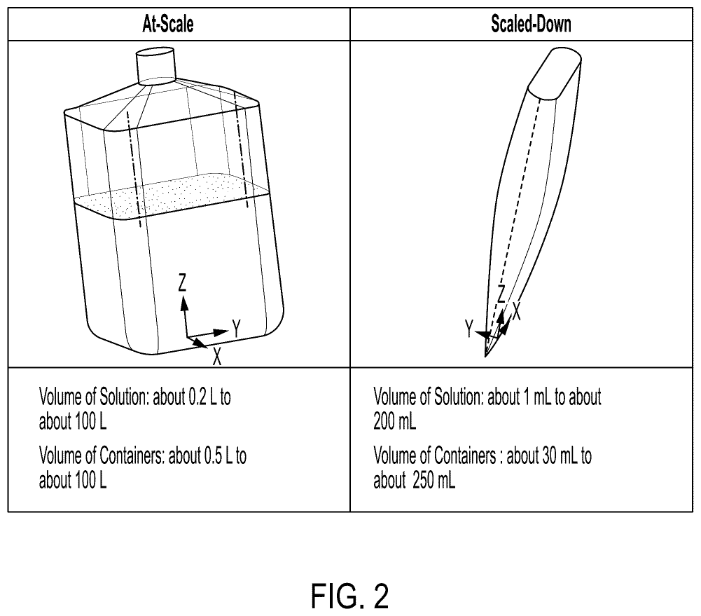

For example, FIG. 1 shows an outline of a computational fluid dynamics model of the present disclosure according to an exemplary embodiment. A system of equations that represents the physics of material inputs subjected to freeze-thaw operating conditions can govern the computational fluid dynamics model of the present disclosure. User inputs can provide information about the material inputs within freeze-thaw operating conditions to a computational fluid dynamics model of the present disclosure. Information about the material inputs subjected to freeze-thaw operating conditions can include the geometry of a container and the mechanical and thermal properties of the material inputs, including, but not limited to, any and all parts of the composition formulation. For example, FIG. 2 shows a representation of an at-scale solution (e.g., about 0.2 L to about 100 L) in an at-scale container (e.g., about 0.5 L to about 100 L) with a rectangular cuboid geometry within a computational fluid dynamic model of the present disclosure according to an exemplary embodiment. FIG. 2 also shows a representation of a scaled-down solution in a scaled-down container (e.g., about 30 mL to about 250 mL) with a swollen bag geometry within a computational fluid dynamics model of the present disclosure according to an exemplary embodiment. User inputs can also provide information about freeze-thaw operating conditions to a computational fluid dynamics model of the present disclosure. Information about freeze-thaw operating conditions can include temperatures, convection modes, adjacent airflows, densities, and proximities of containers to one another.

A computational fluid dynamics model of the present disclosure can, for example, be modeled using the fluid simulation software ANSYS Fluent (ANSYS, Inc., Cannonsburg, Pennsylvania). For a comprehensive overview of modeling options compatible with a computational fluid dynamics model of the present disclosure available within ANSYS Fluent, see ANSYS Fluent Theory Guide, Release 2021 R1, January 2021, ANSYS, Inc., the entire teachings of which are herein incorporated, and ANSYS Fluent User's Guide, Release 2021 R1, January 2021, ANSYS, Inc., the entire teachings of which are herein incorporated. The system of equations that governs a computational fluid dynamics model of the present disclosure can, for example, model mass transfer and heat transfer. Equations included in the governing system can, for example, include the Navier-Stokes equations, a mass conservation equation, momentum conservation equations, an energy conservation equation, an energy equation, turbulence equations, species equations, an equation for back diffusion, and an equation modeling thermal and solutal buoyancy. A shell conduction method can, for example, be used to model the material of the boundaries imposed by a container to account for the effect of the material of the container without solving for the material structure. A mushy zone approximation method can, for example, be used to model the freeze-front within containers so that the freezing of the liquid is advanced spatially based on the modeled freezing operating conditions. Multiphase flow simulations can, for example, be used to model stagnant air between the fill level of a solution and upper boundaries of containers. A volume of the fluid method can, for example, be used to model the air-liquid interface. An interfacial anti-diffusion surface model can, for example, be used to account for the significant difference between the viscosity of the air and the solution.

A computational fluid dynamics model of the present embodiment can use information about the material inputs and freeze-thaw operating conditions to determine solutions to the selected system of equations. Solutions to the governing system of equations can provide physical measures of freeze-thaw processes within a container subjected to freeze-thaw operating conditions. However, it may be difficult to determine analytical solutions to the selected system of equations. Instead, a computational fluid dynamics model of the present disclosure can determine numerical solutions that approximate the analytical solutions to the selected system of equations using spatial and temporal discretization.

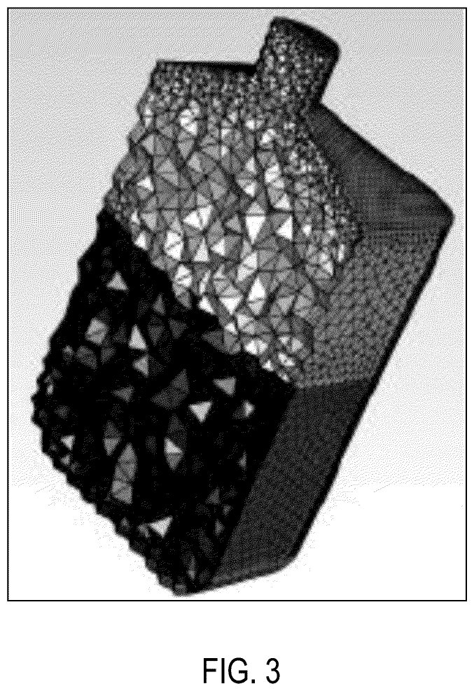

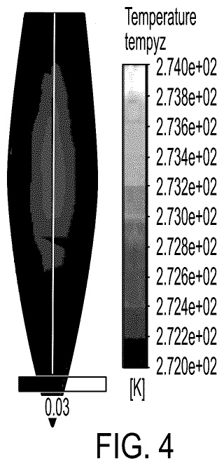

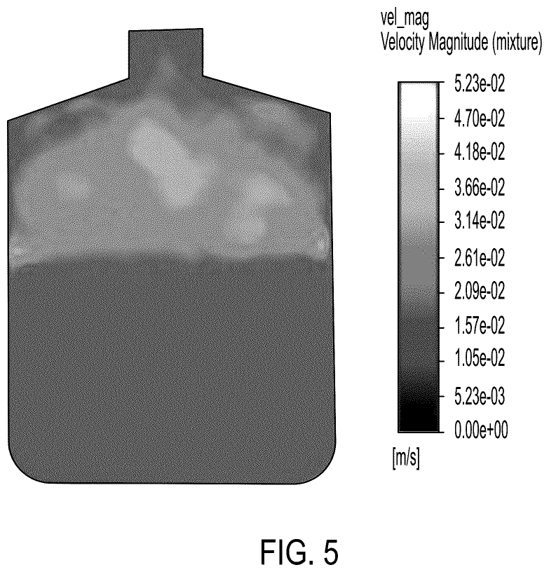

Spatial discretization partitions the spatial domain within a container into discrete volumes at a point while the container is subjected to freeze-thaw operating conditions. FIG. 3 shows the spatial domain of an at-scale container partitioned into discrete volumes after spatial discretization at a point while the container is subjected to freezing operating conditions according to an exemplary embodiment. A computational fluid dynamics model of the present disclosure can determine numerical solutions that approximate (e.g., predict) the average values of analytical solutions within each discrete volume of the discretized spatial domain within a container (e.g. location-specific). For example, FIG. 4 shows the predicted thermodynamic temperatures of a scaled-down solution in a scaled-down container at a point while the scaled-down container is subjected to freezing operating conditions according to an exemplary embodiment. Additionally, FIG. 5 shows the predicted velocities within an at-scale container partially filled with an at-scale solution at a point while the at-scale container is subjected to freezing operating conditions according to an exemplary embodiment. Thus, a computational fluid dynamics model of the present disclosure can predict physical measures of freeze-thaw processes for the entire spatial domain within containers subjected to freeze-thaw operating conditions across scales and geometries.

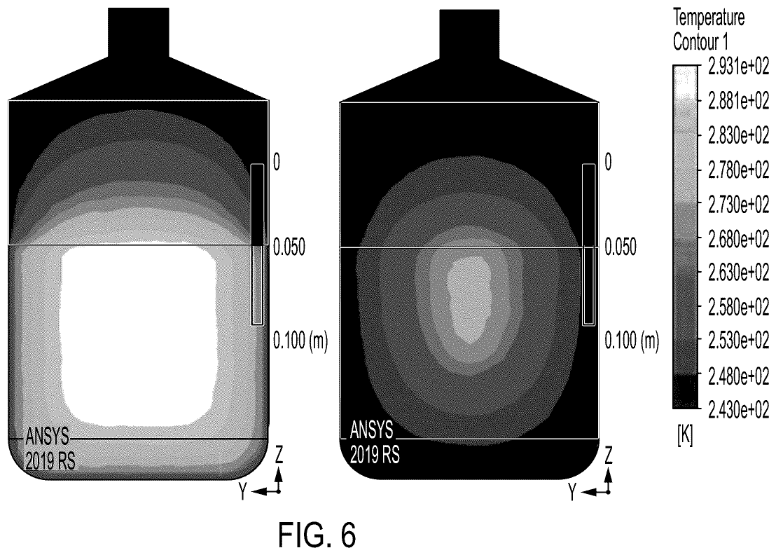

Temporal discretization performs spatial discretization at regular intervals while a container is subjected to freeze-thaw operating conditions. A computational fluid dynamics model can approximate the analytical solutions at time points between sequential spatial discretization operations using interpolation. For example, FIG. 6 shows the predicted thermodynamic temperatures within an at-scale solution in an at-scale container at two points while the at-scale container is subjected to freezing operating conditions according to an exemplary embodiment. Thus, a computational fluid dynamics model of the present disclosure can approximate (e.g., predict) physical measures of freeze-thaw processes within a container subjected to freeze-thaw operating conditions across the entire spatiotemporal domain.

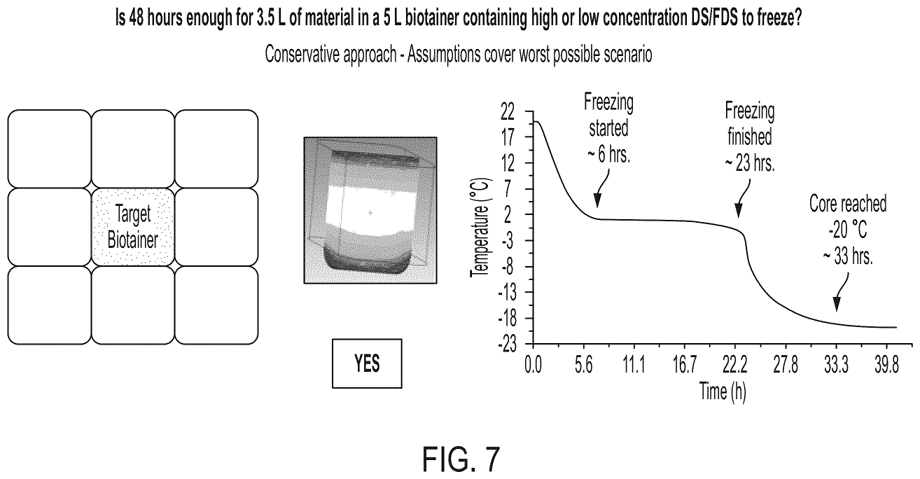

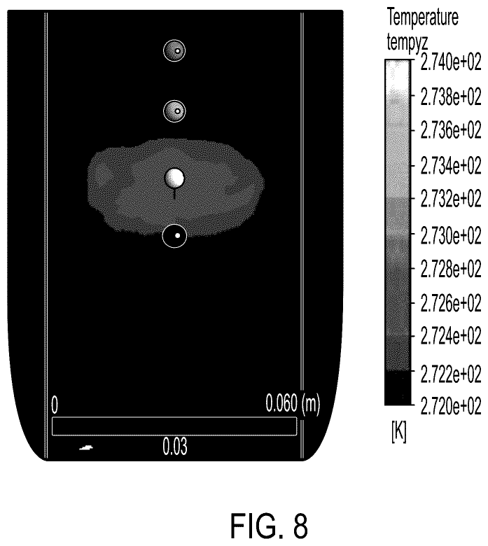

Numerical solutions determined by a computational fluid dynamics model of the present disclosure can predict the thermodynamic temperatures of solutions within containers subjected to freeze-thaw operating conditions. For example, FIG. 4-6 show the thermodynamic temperatures of solutions predicted by a computational fluid dynamics model of the present disclosure if subjected to the modeled freezing operating conditions according to an exemplary embodiment. The thermodynamic temperatures of solutions subjected to freeze-thaw operating conditions can be used to determine the total freeze-thaw time, the first point to freeze, the first point to thaw, the last point to freeze, the last point to thaw, the time at which the center freezes, the time at which the center thaws, and the temperature profile at the edge of the solution. For example, FIG. 7 shows a computational fluid dynamics model of the present disclosure can predict whether 48 hours is enough time for freezing operating conditions to freeze 3.5 L of a high or low concentration drug substance/free drug substance solution in a 5 L Polycarbonate Bottle. User inputs can provide the computational fluid dynamics model shown in FIG. 7 with information about the material inputs and freezing operating conditions, including the position of the 5 L Polycarbonate Bottle relative to other bottles within the freezing operating conditions. The computational fluid dynamics model shown in FIG. 7 can make conservative predictions by modeling the theoretical last point of a solution to freeze (i.e., the center of the solution). Additionally, FIG. 8 shows the last point to freeze (e.g., the red sphere) in a scaled-down solution subjected to freezing operating conditions predicted by a computational fluid dynamics model of the present disclosure according to an exemplary embodiment. Numerical solutions determined by a computational fluid dynamics model of the present disclosure can also approximate the freeze-thaw rates of a solution subjected to freeze-thaw operating conditions.

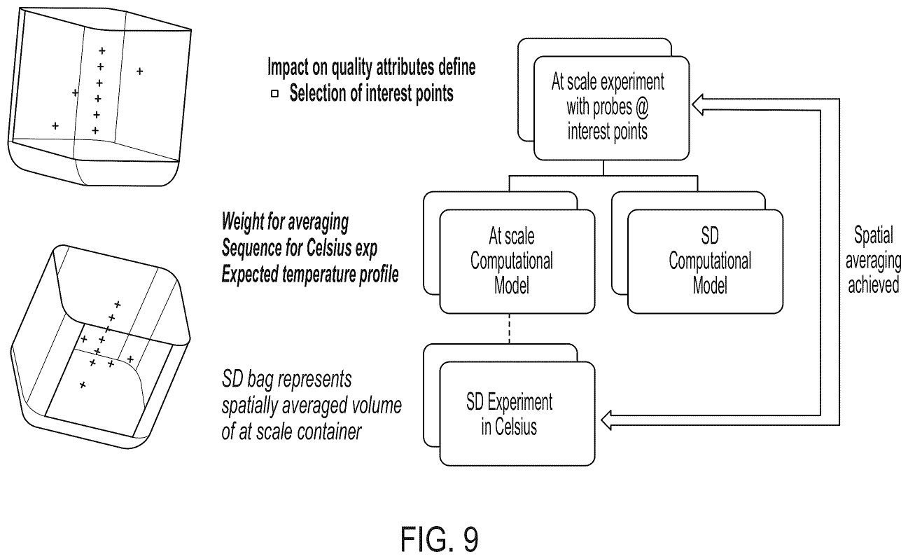

Spatially weighting the predicted physical measures of a solution subjected to freeze-thaw operating conditions and interpolating can provide the average physical measures while the solution is subjected to freeze-thaw operating conditions. For example, FIG. 9 indicates that a computational fluid dynamics model of the present disclosure can predict average representations of at-scale solutions subjected to freeze-thaw operating conditions, including the averaged temperatures, using weighted averaging according to an exemplary embodiment. Furthermore, weighted averaging enables the computational fluid dynamics model of the present disclosure to model the freeze-thaw processes of solutions across scales and geometries. For example, FIG. 2 shows representations of an at-scale solution in an at-scale container with a rectangular cuboid geometry and a scaled-down solution in a scaled-down container with a swollen bag geometry within a computational fluid dynamics model of the present disclosure according to an exemplary embodiment. Additionally, FIGS. 5-7 show physical measures of at-scale solutions in at-scale containers with longitudinal cuboid geometries that were predicted by computational fluid dynamics models of the present disclosure according to exemplary embodiments. In contrast, FIG. 4 and FIG. 8 show physical measures of scaled-down solutions in scaled-down containers with a swelled bag geometry that were predicted by computational fluid dynamics models of the present disclosure according to exemplary embodiments.

In some embodiments, the average physical measures of solutions subjected to freeze-thaw operating conditions can be freeze-thaw profiles. In some embodiments, freeze-thaw profiles can include the predicted average temperatures and total freeze-thaw times of solutions subjected to freeze-thaw operating conditions. FIG. 9 also indicates that the computational fluid dynamics model of the present disclosure can represent the averaged volumes of at-scale solutions subjected to freeze-thaw operating conditions in scaled-down solutions by developing set-temperature sequences. In some embodiments, a scaled-down solution subjected to a set-temperature sequence developed by the computational fluid dynamics model reproduces the predicted average temperature profile (e.g., the predicted freeze-thaw profile) of the at-scale solution in the scaled-down solution.

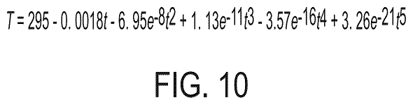

In some embodiments, the computational fluid dynamics model can develop a set-temperature sequence by first fitting an equation to the predicted freeze-thaw profile of an at-scale solution. FIG. 10 presents an exponential equation fit to the average thermodynamic temperatures of a freezing profile for an at-scale solution by the computational fluid dynamics model of the present disclosure according to an exemplary embodiment. In some embodiments, the computational fluid dynamics model can then set the equation as a transient temperature boundary condition when predicting a set-temperature sequence and the freeze-thaw profile of a scaled-down solution subjected to the set-temperature sequence. In one aspect, the computational fluid dynamics model of the present disclosure may refine a set-temperature sequence prediction until the predicted total freeze-thaw time of a scaled-down solution does not differ from the predicted total freeze-thaw time of the at-scale solution by more than about 10%. In another aspect, the computational fluid dynamics model of the present disclosure may refine a set-temperature sequence prediction until the predicted average temperatures of a scaled-down solution do not differ from the predicted average temperatures of the at-scale solution at any point during freeze-thaw processes by more than about 10%.

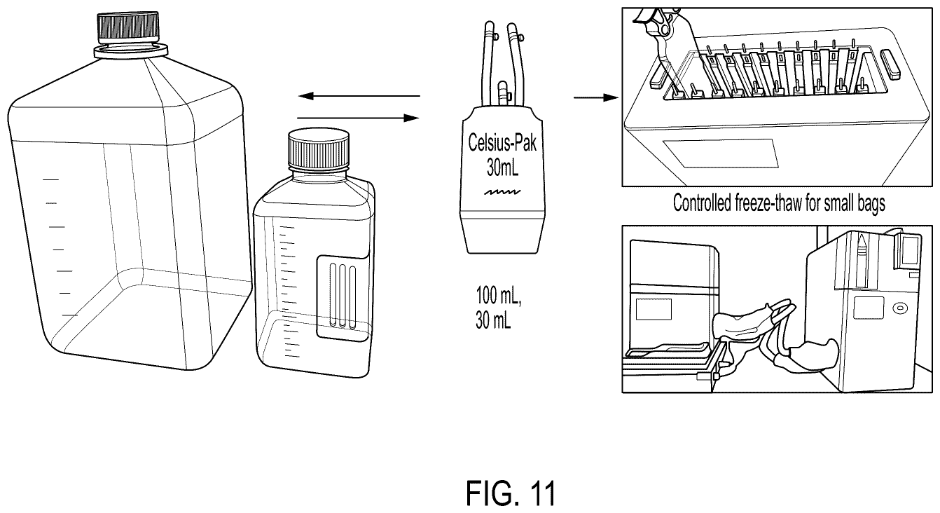

In some embodiments, a scaled-down solution can be subjected to a set-temperature sequence that is predicted to recapitulate the freeze-thaw profile of the at-scale solution. In one aspect, the computational fluid dynamics model can operate a temperature regulation system to produce a set-temperature sequence. In an exemplary embodiment depicted in FIG. 11, the computational fluid dynamics model can operate a Celsius® S3 Benchtop Freezing Platform (Sartorius Stedim Biotech GmbH, Göttingen, Germany) to recapitulate the freeze-thaw profile of an at-scale solution within a 1 L or 5 L Polycarbonate Bottle (Thermo Fisher Scientific, Waltham, Massachusetts) in the scaled-down solution within a 30 mL bag. In one aspect, the temperatures of at least one point of interest within a scaled-down solution can be measured during a freeze-thaw process. In another aspect, the measured temperatures of at least one point of interest in a scaled-down solution can be measured while the scaled-down solution is subjected to a set-temperature sequence. In yet another aspect, the measured temperatures of at least one point of interest in a scaled-down solution subjected to a set-temperature sequence can be used to determine the average temperatures of the scaled-down solution while subjected to a set-temperature sequence. In another aspect, the average temperatures of a scaled-down solution subjected to a set-temperature sequence can be used to determine the total freeze-thaw time, and the actual freeze-thaw profile of the scaled-down solution can comprise the total freeze-thaw time and average temperatures of the scaled-down solution subjected to a set-temperature sequence.

In one aspect, the at-scale solution modeled by the computational fluid dynamics model can be subjected to the modeled freeze-thaw operating conditions while measuring the temperatures of at least one point of interest. In another aspect, the measured temperatures of the at least one point of interest in an at-scale solution subjected to a set of freeze-thaw operating conditions can be used to determine the average temperatures of the at-scale solution. In yet another aspect, the average temperatures of the at-scale solution during a freeze-thaw process can be used to determine the total freeze-thaw time of the solution, and the actual freeze-thaw profile of the at-scale solution can comprise the total freeze-thaw time and average temperatures of the at-scale solution subjected to a set of freeze-thaw operating conditions.

In one aspect, the total freeze-thaw times of the actual and predicted at-scale and scaled-down freeze-thaw profiles may not differ from one another by more than about 10%. In another aspect, the average temperatures of the actual and predicted at-scale and scaled-down freeze-thaw profiles at a point during a freeze-thaw process may not differ from one another by more than about 10%. FIG. 2 presents an exemplary embodiment in which the actual and predicted total freeze-thaw times of an at-scale solution in a 5 L Polycarbonate Bottle and the scaled-down solution in a 100 mL bag can differ by less than about 6.67%.

The present disclosure provides the benefit of determining the effects of at-scale freeze-thaw rates on the quality attributes of freeze-thaw sensitive substances without performing at-scale experiments. Accordingly, in one aspect, at least one quality attribute of the scaled-down volume can be determined after a freeze-thaw process. A quality attribute of the present disclosure can be a physical, chemical, biological, or microbiological property or characteristic that should be within an appropriate limit, range, or distribution to ensure the desired product quality.

For example, in one aspect, a solution can comprise a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody. Therefore, the method of the present disclosure provides a robust and cost-effective method for optimizing at-scale pharmaceutical freeze-thaw processes to ensure the quality, safety, and efficacy of a pharmaceutical for a patient.

### Shell Conduction

In some embodiments, a shell conduction method can model the material of the boundaries imposed by a container to account for the effect of the material of the container without solving for the material structure. The shell conduction method can account for thermal mass in transient thermal analysis problems. The shell conduction method can also allow heat conduction through multiple junctions. The shell conduction method can be applied to the boundary and internal walls. Using a fluid simulation software, such as, ANSYS Fluent (ANSYS, Inc., Cannonsburg, Pennsylvania), the shell conduction method can model thermal conduction in the planar and normal direction of the boundaries imposed by the walls. The shell conduction method can model thin sheets without needing to mesh the wall thickness in a preprocessor. A user can switch the conjugate heat transfer of any wall on or off when the shell conduction method is utilized. Specifying a thickness for the wall boundaries, a material property, and enabling the shell conduction method in ANSYS Fluent can cause ANSYS Fluent to grow a layer of prism cells or hex cells for the wall, depending on the type of face mesh that is utilized. Modeling the boundaries imposed by the walls without a shell conduction method and without specifying a thickness for the boundaries imposed by the container may cause the material of containers to present no thermal resistance to heat transfer in said model. Alternatively, specifying a thickness for the boundaries imposed by the container without the shell conduction method may only produce the appropriate thermal resistance across the boundaries imposed by the container in the normal direction.

### At-Scale Containers

In some aspects, the computational fluid dynamics model can predict location-specific physical quantities of freeze-thaw processes for the entire spatial domain within an at-scale container with a volume that is between about 1 L and about 20 L across geometries. In some aspects, the computational fluid dynamics model can predict location-specific physical quantities of freeze-thaw processes for an at-scale solution that is between about 0.75 L and about 15 L across geometries.

Exemplary, non-limiting at-scale containers for which the computational fluid dynamics model can predict location-specific physical quantities of freeze-thaw processes for the entire spatial domain within the container include a 1 L Polycarbonate Bottle, a 2 L Polycarbonate, a 5 L Polycarbonate Bottle, a 10 L Polycarbonate Bottle, a 20 L Polycarbonate Bottle, a 1 L bag, a 2 L bag, an 8.3 L bag, and a 16.6 L bag. Exemplary, non-limiting at-scale container geometries for which the computational fluid dynamics model can predict location-specific physical quantities of freeze-thaw processes for the entire spatial domain within the container. In one aspect, the at-scale container is a square geometry container and in one aspect, the scaled-down container is a bag geometry container in which a solution inside the bag causes swelling.

### Scaled-Down Containers

In some aspects, the computational fluid dynamics model can predict location-specific physical quantities of freeze-thaw processes for the entire spatial domain within a scaled-down container with a volume that is between about 30 mL and about 100 mL across geometries. In some aspects, the computational fluid dynamics model can predict location-specific physical quantities of freeze-thaw processes for a scaled-down solution that is between about 20 mL and about 100 mL across geometries.

Exemplary, non-limiting scaled-down containers for which the computational fluid dynamics model can predict location-specific physical quantities of freeze-thaw processes for the entire spatial domain within the container include a 30 mL bag and a 100 mL bag. Exemplary, non-limiting scaled-down container geometries for which the computational fluid dynamics model can predict location-specific physical quantities of freeze-thaw processes for the entire spatial domain within the container include a bag geometry in which a solution inside the bag causes swelling.

The present invention will be more fully understood by referencing the following examples. They should not, however, be construed as limiting the scope of the present invention.

### Example 1

Temperature probes monitored the temperature of at-scale volumes of solutions at multiple points of interest throughout freeze-thaw processes to determine the freezing rates. Information about the freeze-thaw rates informed a computational fluid dynamics (CFD) model of freeze-thaw systems. The CFD model was designed to provide location-specific predictions for the freeze-thaw rates of a solution at every location within a container across scales and geometries. The CFD model was also designed to control a temperature regulation system so that the scaled-down freeze-thaw rates of a solution mimic the at-scale freeze-thaw rates.

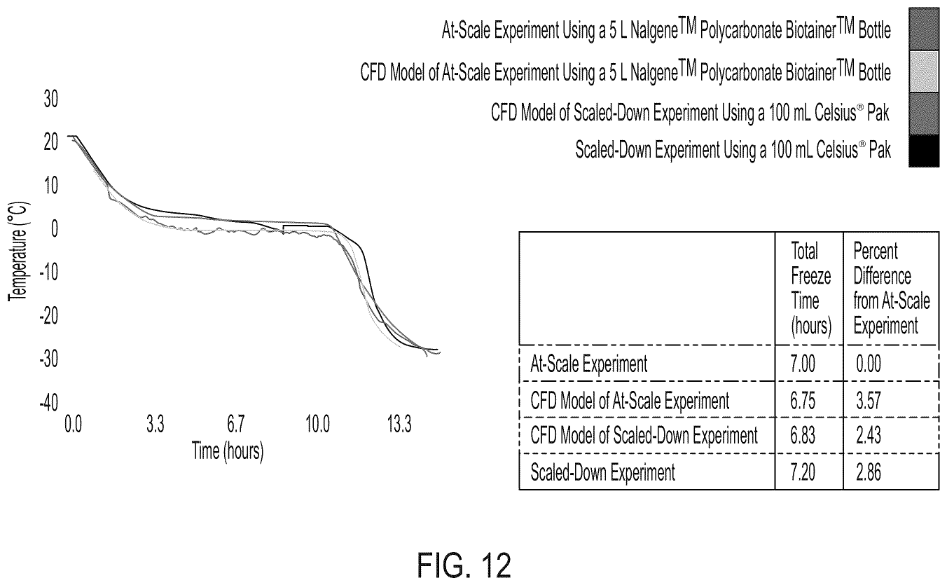

The CFD model was validated, in part, by comparing the total freeze time of an at-scale solution in a 5 L Nalgene™ Polycarbonate Biotainer™ Bottle to the total freeze time of the at-scale solution in a 5 L Nalgene™ Polycarbonate Biotainer™ Bottle predicted by the CFD model (e.g., “the predicted at-scale freezing profile”) as seen in FIG. 12. The predicted (e.g., 6.75 hours) and actual (e.g., 7 hours) total freeze times of the at-scale volume of the solution differed by 3.57% (e.g., “the at-scale experiment”), as seen in FIG. 12. The CFD model then fit an equation to the predicted average thermodynamic temperatures in the at-scale freezing profile and set the equation as a transient temperature boundary condition. Next, the CFD model simultaneously predicted the freezing profile of the scaled-down solution in a 100 mL Celsius® Pak subjected to a set-set-temperature sequence (e.g., “the predicted scaled-down freezing profile) and developed the set-set-temperature sequence so that the predicted scaled-down freezing profile mimicked the predicted at-scale freezing profile. The CFD model then produced a scaled-down input that could manipulate the set temperature of a Celsius® S3 Benchtop Freezing Platform in accordance with the developed temperature progression scheme, as seen in FIG. 11. Subsequently, the CFD model was further validated by freezing the scaled-down volume of the solution in a 100 mL Celsius® Pak using a Celsius® S3 Benchtop Freezing Platform manipulated by the scaled-down input and comparing the predicted and actual total freeze times of the scaled-down solution to the actual total freeze time of the at-scale volume of the solution. The predicted (e.g., 6.83 hours) and actual (e.g., 7.2 hours) total freeze times of the scaled-down volume of the solution differed from the actual total freeze time of the at-scale volume of the solution by 2.43% and 2.86%, respectively, as seen in FIG. 12.

The CFD model included a governing system of equations with simplifying assumptions to mathematically describe the physics underlying the freezing systems, as seen in FIG. 1. The system of equations modeled mass transfer, heat transfer, and the properties of materials within the freezing systems. User inputs provided information about the material inputs and operating conditions to the CFD model equations, as seen in FIG. 1. Information about the materials included the geometry of the containers and the mechanical properties and thermal properties of the material inputs, as seen in FIG. 1. The information about the operating conditions included the temperature, the convection mode, the adjacent airflow mechanism, and the proximity of a container to other containers within the freezing operating conditions, as seen in FIG. 1. Numerical solutions approximated the continuous solutions to the differential equations within the system of equations governing the CFD model using spatial discretization according to a time advancement scheme, as seen in FIG. 1. Spatial discretization spatially averaged regions within a container for the entire spatial domain at a discrete time during simulations of the freezing processes, as seen in FIG. 1. The spatially averaged regions provided approximations of the continuous solutions to the differential equations at a discrete time, as seen in FIG. 1.

Temporal spatialization performed sequential spatial discretization techniques for the span of the temporal domain for simulations of the freezing processes, as seen in FIG. 1. Consequently, the CFD model could provide location-specific approximations of the physical quantities of the freezing processes, including the freeze-thaw rates, at any point within the containers after receiving information about the freezing systems from the user inputs. Weighted spatial averaging inside the freezing domain enabled the CFD model to scale down an average representation of the bulk volume of the 5 L Nalgene™ Polycarbonate Biotainer™ Bottle into a 100 mL Celsius® Pak, as seen in FIG. 9. Thus, the 100 mL Celsius® Pak was an average representation of the bulk volume of the 5 L Nalgene™ Polycarbonate Biotainer™ Bottle.

A shell conduction method modeled the 5 L Nalgene™ Polycarbonate Biotainer™ Bottle material and the 100 mL Celsius® Pak material to account for the effect of the bottle material without the extensive cost of solving for the material structure. The 100 mL Celsius® Pak model containing the solution reflected swelling that occurred due to the solution, as well as the softer convective heat transfer gradients across the sides and bottom to mimic the heat transfer rates of the 5 L Nalgene™ Polycarbonate Biotainer™ Bottle. The shell conduction method can account for thermal mass in transient thermal analysis problems. The shell conduction method can also allow heat conduction through multiple junctions. The shell conduction method can be applied to the boundary walls and internal walls. Using the fluid simulation software ANSYS Fluent, the shell conduction method can model thermal conduction in the planar and normal direction of the boundaries imposed by the walls. The shell conduction method can model thin sheets without needing to mesh the wall thickness in a preprocessor. A user can switch the conjugate heat transfer of any wall on or off when the shell conduction method is utilized. Specifying a thickness for the wall boundaries, a material property, and enabling the shell conduction method in ANSYS Fluent can cause ANSYS Fluent to grow a layer of prism cells or hex cells for the wall, depending on the type of face mesh that is utilized. Modeling the boundaries imposed by the walls without a shell conduction method and without specifying a thickness for the boundaries imposed by the container may cause the material of containers to present no thermal resistance to heat transfer. Alternatively, specifying a thickness for the boundaries imposed by the container without the shell conduction method may only produce the appropriate thermal resistance across the boundaries imposed by the container in the normal direction.

ANSYS Fluent modeled the freeze-front using a mushy zone approximation method available within ANSYS Fluent so that liquid freezing advanced spatially based on the freezing operating conditions. Multiphase flow simulations modeled the air between the solution fill level and the cap of the 5 L Nalgene™ Polycarbonate Biotainer™ Bottle. The volume of fluid method models the air-liquid interface, and an interfacial anti-diffusion surface model accounts for the significant difference between the viscosity of the air and liquid.

### Example 2

The CFD model approximated whether 3.5 L of solution with a high or low concentration of a drug substance or a free drug substance (e.g., user-specified) within a 5 L Nalgene™ Polycarbonate Biotainer™ Bottle could freeze within 48 hours, as seen in FIG. 7. A user provided the CFD model with information about the material inputs (e.g., the geometry of the 5 L Nalgene™ Polycarbonate Biotainer™ Bottle, and the mechanical and thermal properties of the material inputs). A user provided the CFD model with information about the freezing operating conditions, including the position of the 5 L Nalgene™ Polycarbonate Biotainer™ Bottle relative to other bottles within the freezing operating conditions. The CFD model took a conservative approach and determined whether 48 hours would be enough time for the center of the solution (e.g., the theoretical last point to freeze) to freeze. Given the user-provided information about the material inputs and freezing operating conditions (not specified), the computational fluid dynamics model approximates that the solution will freeze within 48 hours.

What is claimed is:

1. A method for freezing a solution, comprising:
(a) using a computational fluid dynamics model to predict a first freezing profile of an at-scale volume of the solution subjected to first freezing operating conditions, wherein the first freezing profile includes predicted average temperatures of the solution during freezing and total freeze time;
(b) using the computational fluid dynamics model to fit a transient temperature boundary equation to the first freezing profile;
(c) using the computational fluid dynamics model to predict a set-temperature sequence that produces a predicted second freezing profile of the scaled-down volume of the solution, wherein:
(i) the transient temperature boundary equation is a condition for predicting the set-temperature sequence; and
(ii) the second freezing profile includes predicted average temperatures of the solution during freezing and total freeze time; and
(d) freezing the scaled-down volume of the solution using the set-temperature sequence.

2. The method of claim 1, further comprising determining at least one quality attribute of said scaled-down volume of the solution after freezing.

3. The method of claim 2, wherein said solution comprises a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody.

4. The method of claim 3, further comprising operating a temperature regulation system using said computational fluid dynamics model to produce said set-temperature sequence for freezing said scaled-down volume.

5. The method of claim 4, further comprising measuring a temperature of at least one point of interest in said scaled-down volume throughout freezing.

6. The method of claim 5, wherein said at-scale volume is between about 0.2 L and about 20 L.

7. The method of claim 6, wherein said scaled-down volume is between about 20 mL and about 100 mL.

8. The method of claim 5, wherein said at-scale volume is in an at-scale container with a volume of between about 1 L and about 20 L.

9. The method of claim 8, wherein said scaled-down volume is in a scaled-down container with a volume of between about 30 mL and about 100 mL.

10. The method of claim 9, wherein the at-scale container is selected from a group comprising a 1 L polycarbonate bottle, a 2 L polycarbonate bottle, a 5 L polycarbonate bottle, a 10 L polycarbonate bottle, a 20 L polycarbonate bottle, a 1 L bag, a 2 L bag, an 8.3 L bag, and a 16.6 L bag.

11. The method of claim 10, wherein the scaled-down container is selected from a group comprising a 30 mL bag and a 100 mL bag.

12. A method for thawing a solution, comprising:
(a) using a computational fluid dynamics model to predict a first thawing profile of an at-scale volume of the solution subjected to first thawing operating conditions, wherein the first thawing profile includes predicted average temperatures of the solution during thawing and total thaw time;
(b) using the computational fluid dynamics model to fit a transient temperature boundary equation to the first thawing profile;
(c) using the computational fluid dynamics model to predict a set-temperature sequence that produces a predicted second thawing profile of the scaled-down volume of the solution, wherein:
(i) the transient temperature boundary equation is a condition for predicting the set-temperature sequence; and
(ii) the second thawing profile includes predicted average temperatures of the solution during thawing and total thaw time; and
(d) thawing the scaled-down volume of the solution using the set-temperature sequence.

13. The method of claim 12, further comprising determining at least one quality attribute of said scaled-down volume of the solution after thawing.

14. The method of claim 13, wherein said solution comprises a pharmaceutical, a pharmaceutical product, a drug, a chemical compound, a nucleic acid, a toxin, a peptide, a protein, a fusion protein, an antibody, an antibody fragment, a Fab region of an antibody, an antibody-drug conjugate, a biopharmaceutical, a pharmaceutical protein product, or an antibody.

15. The method of claim 14, further comprising operating a temperature regulation system using said computational fluid dynamics model to produce said set-temperature sequence for thawing said scaled-down volume.

16. The method of claim 15, further comprising measuring a temperature of at least one point of interest in said scaled-down volume throughout thawing.

17. The method of claim 16, wherein said at-scale volume is between about 0.75 L and about 15 L.

18. The method of claim 17, wherein said scaled-down volume is between about 20 mL and about 100 mL.

19. The method of claim 16, wherein said at-scale volume is in an at-scale container with a volume of between about 1 L and about 20 L.

20. The method of claim 19, wherein said scaled-down volume is in a scaled-down container with a volume of between about 30 mL and about 100 mL.

21. The method of claim 20, wherein the at-scale container is selected from a group comprising a 1 L polycarbonate bottle, a 2 L polycarbonate bottle, a 5 L polycarbonate bottle, a 10 L polycarbonate bottle, a 20 L polycarbonate bottle, a 1 L bag, a 2 L bag, an 8.3 L bag, and a 16.6 L bag.

22. The method of claim 21, wherein the scaled-down container is selected from a group comprising a 30 mL bag and a 100 mL bag.

23. The method of claim 1, wherein the computational fluid dynamics model accounts for environmental factors.

24. The method of claim 23, wherein the environmental factors are selected from the group comprising airflow, proximity to other surfaces of varying temperatures, relative humidity, pressure, and any combinations thereof.

25. A method for freezing a solution, comprising:
(a) using a computational fluid dynamics model to predict a freezing profile of the at-scale solution subjected to freezing operating conditions;
(b) determining freezing will occur within a necessary period of time; and
(c) freezing the at-scale volume of the solution using the freezing operating conditions.

26. A method for thawing a solution, comprising:
(a) using a computational fluid dynamics model to predict a thawing profile of the at-scale solution subjected to first thawing operating conditions;
(b) determining thawing will occur within a necessary period of time; and
(c) thawing the at-scale volume of the solution using the thawing operating conditions.
